(19) United States
(12) Patent Application Publication
(10) Pub. No.: US 2022/0257717 A1
(43) Pub. Date: Aug. 18, 2022

(54) Methods of Treatment Using Encapsulated Cells

(71) Applicant: PrimeGen Biotech, LLC, Santa Ana, CA (US)

(72) Inventors: Joel Marh, Santa Ana, CA (US); Julia Kim, Santa Ana, CA (US)

(21) Appl. No.: 17/626,764

(22) PCT Filed: Jul. 10, 2020
(86) PCT No.: PCT/US2020/041580

§ 371 Date: Jan. 12, 2022

Related U.S. Application Data
(60) Provisional application No. 62/873,365, filed on Jul. 12, 2019.

(51) Int. Cl. A61K 38/18; C07K 14/51; C12N 5/00; C12N 5/071

(52) U.S. Cl. CPC A61K 38/1875; C07K 14/51; C12N 2510/00; C12N 5/0671; C12N 2501/155; C12N 5/0012

(57) ABSTRACT

Provided are methods of treatment comprising administering to a subject in need thereof a therapeutically effective amount of encapsulated cells to an affected tissue or organ.

### TECHNICAL FIELD

This disclosure relates to systems, devices, methods, and compositions for treating tissues and organs using administration of encapsulated cells. More specifically, described herein are methods to treat fibrotic organs including, for example, the kidney and liver.

### BACKGROUND

Various conditions are associated with the degradation of tissues in the body. Both the tissues themselves, as well as organs, may atrophy and eventual fail unless appropriately treated and regenerated. These conditions can include diseases such as fibrosis.

Fibrosis is a pathogenic condition characterized by an excessive accumulation and deposition of extracellular matrix (ECM), immunocomponents, and connective or scar tissue, in and around organs, joints, and/or other bodily tissues. Fibrosis can lead to scarring, inflammation, or damage and, in some cases, failure of the tissue or organ. Fibrosis can affect any bodily tissue or organ, but can be especially acute in larger solid tissue organs such as the liver, kidney, spleen, lungs and heart. Reduced blood flow to the affected organ(s) can be a major contributor to tissue fibrosis. Another contributing factor to organ failure is reduced cellular energy production, characterized by mitochondrial insufficiency and impaired adenosine triphosphate (ATP) metabolism.

In cases, the affected organs can become compromised to the point where therapeutic intervention cannot reach the target organ in sufficient levels to reverse tissue damage and restore organ function. Thus, “chronic” illnesses such as Chronic Kidney Disease (CKD) or cirrhosis of the liver can lead to progressive organ failure where the only available treatments are organ transplant or life-long dialysis. Organ transplants are expensive, require precise tissue matching, are limited by the number of suitable donor candidates, and require long-term immunosuppressive therapy. Dialysis is extremely burdensome, limiting the patient's ability to travel and necessitating frequent painful, expensive, and time-consuming treatments. Both tissue transplants and dialysis significantly impair life quality for the patent afflicted with CKD.

Cell encapsulation technology refers to immobilization of cells within biocompatible, semipermeable membranes. This technique consists of enclosing the biologically active material within a polymeric matrix surrounded by a semipermeable membrane that is designed to circumvent immune rejection. The capsule membrane allows the bi-directional diffusion of nutrients, oxygen, and waste and the secretion of the therapeutic product. It has the advantage of preventing immune cells and antibodies, which might destroy the enclosed cells, from entering the capsule. The encapsulation of cells instead of therapeutic products allows the delivery of molecules of interest for a longer period of time because cells can release these molecules continuously.

### SUMMARY

The present disclosure is based, at least in part, on the non-limiting theory that encapsulated cells can be used to treat conditions in mammals. For example, the present disclosure provides methods that can prevent, arrest, or reverse tissue and organ damage before the damage is irreparable, and help repair damage which has occurred. Embodiments comprise the production and use of encapsulated cells.

Encapsulated cells as disclosed herein can comprise any cell that releases an active agent, for example cytokines such as Bone morphogenetic protein-7 (BMP7), for example native or recombinant BMP7, which is a protein of the TGF-β super family and increasingly regarded as a counteracting molecule against TGF-β.

In embodiments, the encapsulated cells are transfected cells. In embodiments, Human Embryonic Kidney (HEK) cells are utilized. The encapsulation technology encloses live cells in protective “capsules” about the size of the head of a pin. The capsules are designed to allow blood to enter and nourish the living cells inside them. The live cells thrive while the capsules are in the body. However, the capsules are also designed to protect the live cells from attack by the body's immune system. The live cells are too large to escape from the capsules, and the body's immune system cells are too large to enter the capsules.

Disclosed embodiments comprise the use of human cells to produce BMP7, for example a native BMP7, for example in a target tissue. Embodiments disclosed herein can produce a sustained, consistent amount of BMP7 over time.

Disclosed embodiments can target specific areas in the body, as the short half-life of BMP7 decreases the risk of unwanted peripheral effects and increases the BMP7 effects in targeted areas in the body.

Disclosed embodiments comprise methods of storing encapsulated cells wherein a freeze/thaw cycle does not inhibit protein expression from the cells.

Disclosed embodiments comprise methods of implanting cells, for example “immortal” cells that do not undergo senescence or contact inhibition, into a patient without tumor development.

Disclosed embodiments comprise methods of treating fibrotic diseases.

Disclosed embodiments comprise methods comprising use of encapsulated cells, for example BMP7 cells, in combinatorial therapy to prevent the foreign body response or scarring resulting from medical devices, implants, and surgeries. In embodiments, BMP7 can remove and prevent the extracellular matrices and collagen by producing metalloproteinases and competing for SMAD proteins to prevent fibrosis.

Disclosed embodiments can be used to treat patients without producing non-targeted effects, as demonstrated by the lack of visible affects to mice 7 months after injection.

Disclosed embodiments comprise a “suicide” gene that allows the practitioner to stop the production of BMP7, or kill the cell transfected cells.

### BRIEF DESCRIPTION OF THE DRAWINGS

FIG. 1 shows advantages associated with encapsulation of cytokine-expressing cells.

FIG. 2 depicts a disclosed encapsulation process, as well as cultured encapsulated cells.

FIG. 3 illustrate the BMP7 pathway as it relates to fibrosis.

FIG. 4 depicts the role of BMP7 signaling in liver regeneration and liver fibrosis.

FIG. 5 depicts the BMP7 plasmid design.

FIG. 6 depicts the transfection of HEK293T cells.

FIG. 7 depicts the hygromycin-based clonal selection.

FIG. 8 depicts the primer pair gene sequences used to detect BMP7 in transfected HEK293T cells.

FIG. 9 depicts the DNA sequence of the BMP7 expressing plasmid

FIG. 10 depicts BMP7 PCR data of modified cells v. control cells.

FIG. 11 shows quantitative RT PCR data of modified cells v. control cells

FIG. 12 depicts the sequencing verification of transfected HEK293T cells.

FIG. 13 depicts ELISA results from transfected HEK293T cells.

FIG. 14 depicts clonal selection and expansion

FIG. 15 shows single cell selection of a BMP7 transfected cell.

FIG. 16 shows 9-day antibiotic selection of transfected HEK clones.

FIG. 17 depicts encapsulation of BMP7 producing cells

FIG. 18 depicts IQELISA protein expression outside of the encapsulated transfected HEK293T cells.

FIG. 19 shows IQELISA analysis of secreted BMP7 in media.

FIG. 20 shows the production sequence for BMP7 producing cells.

FIG. 21 depicts downstream signal transduction via BMP canonical SMAD pathway.

FIG. 22 shows injectable-size BMP7 encapsulated cells.

FIG. 23 shows effects of administration of BMP7-producing encapsulated cells.

FIG. 24 depicts several of the interactions common to the cytokine signaling pathway.

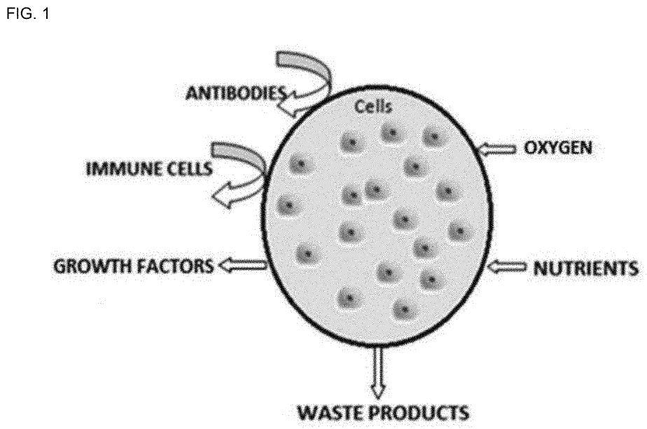

### DETAILED DESCRIPTION

The present disclosure relates to, at least in part, the benefits of treating tissues and organs with encapsulated cells. For example, disclosed methods and compositions can comprise administration of encapsulated cells that produce and release cytokines. Cytokines are small secreted proteins released by cells which have a specific effect on the interactions and communications between cells. Cytokine is a general name; other names include lymphokine (cytokines made by lymphocytes), monokine (cytokines made by monocytes), chemokine (cytokines with chemotactic activities), and interleukin (cytokines made by one leukocyte and acting on other leukocytes). Cytokines can act on the cells that secrete them (autocrine action), on nearby cells (paracrine action), or in some instances on distant cells (endocrine action). There are both pro-inflammatory cytokines and anti-inflammatory cytokines. There is significant evidence showing that certain cytokines/chemokines are involved in not only the initiation but also the persistence of pathologic pain by directly activating nociceptive sensory neurons. Certain inflammatory cytokines are also involved in nerve-injury/inflammation-induced central sensitization, and are related to the development of contralateral hyperalgesia/allodynia.

In embodiments, disclosed devices and methods can comprise methods to reduce or prevent rejection in the body, for example rejection of implants, by limiting or preventing the foreign body fibrosis response that could engulf the encapsulated cells via expression of BMP7.

In embodiments, encapsulated cells as described herein can secret BMP7. BMP7 is a member of the TGF-β superfamily. Like other members of the bone morphogenetic protein family of proteins, it plays a key role in the transformation of mesenchymal cells into bone and cartilage. It is inhibited by noggin and a similar protein, chordin, which are expressed in the Spemann-Mangold Organizer. BMP7 may be involved in bone homeostasis. It is expressed in the brain, kidneys and bladder. BMP7 induces the phosphorylation of SMAD1 and SMAD5, which in turn induce transcription of numerous osteogenic genes. It has been demonstrated that BMP7 treatment is sufficient to induce all of the genetic markers of osteoblast differentiation in many cell types.

BMP7 has been discovered to be crucial in the determination of ventral-dorsal organization in zebrafish. BMP7 causes the expression of ventral phenotypes while its complete inhibition creates a dorsal phenotype. Moreover, BMP7 is eventually (partially) “turned off” in embryonic development in order to create the dorsal parts of the organism

The role of BMP7 in mammalian kidney development is through induction of MET of the metanephrogenic blastema. The epithelial tissue emerging from this MET process eventually forms the tubules and glomeruli of the nephron. BMP7 is also important in homeostasis of the adult kidney by inhibiting epithelial-mesenchymal transition (EMT). BMP7 expression is attenuated when the nephron is placed under inflammatory or ischemic stress, leading to EMT, which can result in fibrosis of the kidney. This type of fibrosis often leads to renal failure, and is predictive of end stage renal disease. BMP7 can also work in the “reverse.” When over activated, BMP7 can remove fibrosis in the kidney via competition for SMAD4 proteins and by reducing TGFb and producing metalloproteinases to remove ECM.

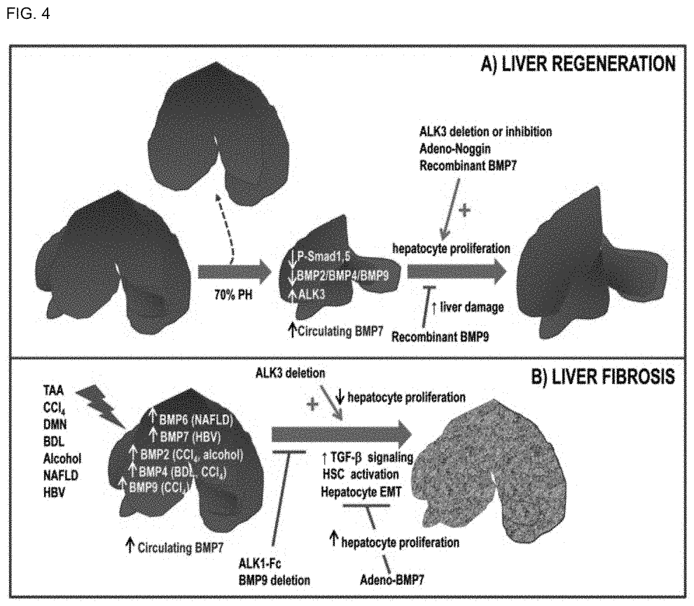

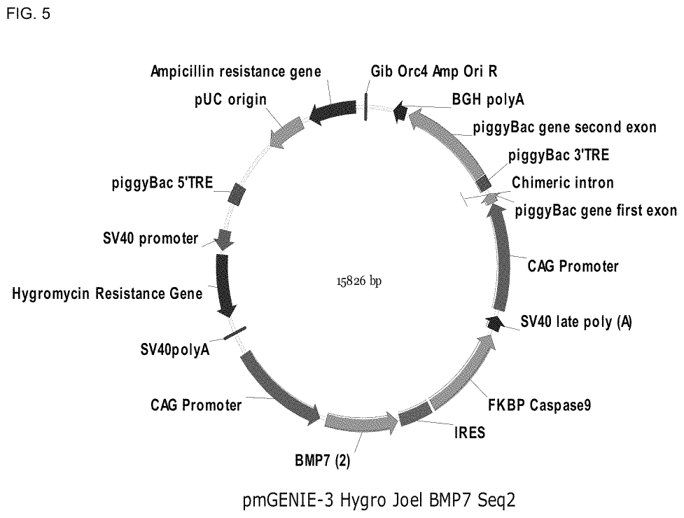

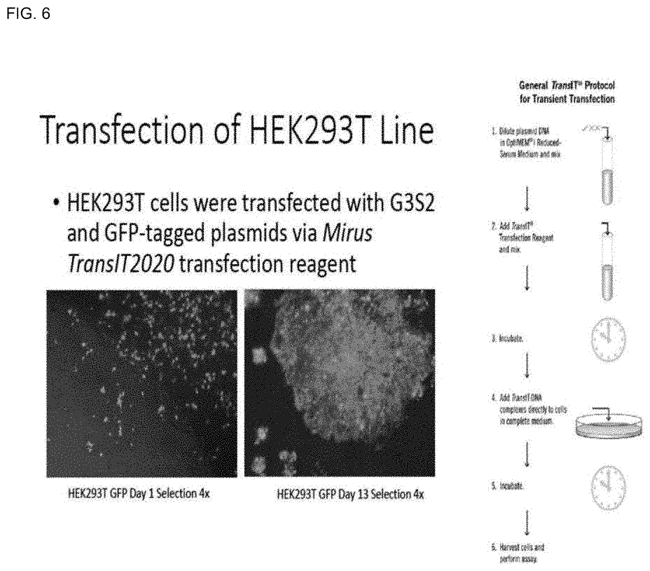

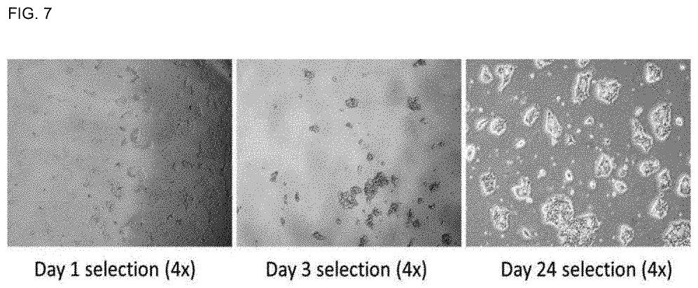

FIG. 4 illustrates the role of BMP signaling in liver regeneration (A) and liver fibrosis (B). (A) Following a partial hepatectomy (PH), BMP signaling is reduced in liver, with a decreased expression of BMP2, BMP4 and BMP9 and a decrease in phospho-Smad1,5,8 (P-Smad1,5) levels. Different approaches show that BMP7, adenovirus-mediated expression of Noggin or ALK3 (BMP receptor type 1A) deletion/inhibition have a pro-regenerative effect in liver, whereas other BMP ligands such as BMP9, increase liver damage; (B) An upregulation of different BMP ligands has been observed in response to liver insults of different nature, such as bile duct ligation, CC14 treatment and others. Accumulating data evidence a protective role for BMP7 in the context of liver fibrosis. Inhibition or deletion of BMP9 has a similar antifibrotic effect, while ALK3 inhibition favors liver fibrosis. The role of other BMPs is currently a matter of study.

In embodiments, cytokine-producing cells are encapsulated, transformed, Human Embryonic Kidney (HEK) cells. Encapsulation of cytokine-expressing cells protects encapsulated cells from host immune system allows cytokines to pass through encapsulated cells into the body, allows for oxygen, waste, and nutrient exchange to support encapsulated cells, and enables the production of cytokines in one area. BMP7 can also prevent the foreign body response (Fibrotic cyst formation) on the encapsulated cells that can interrupt the nutrient and blood supply to the cells.

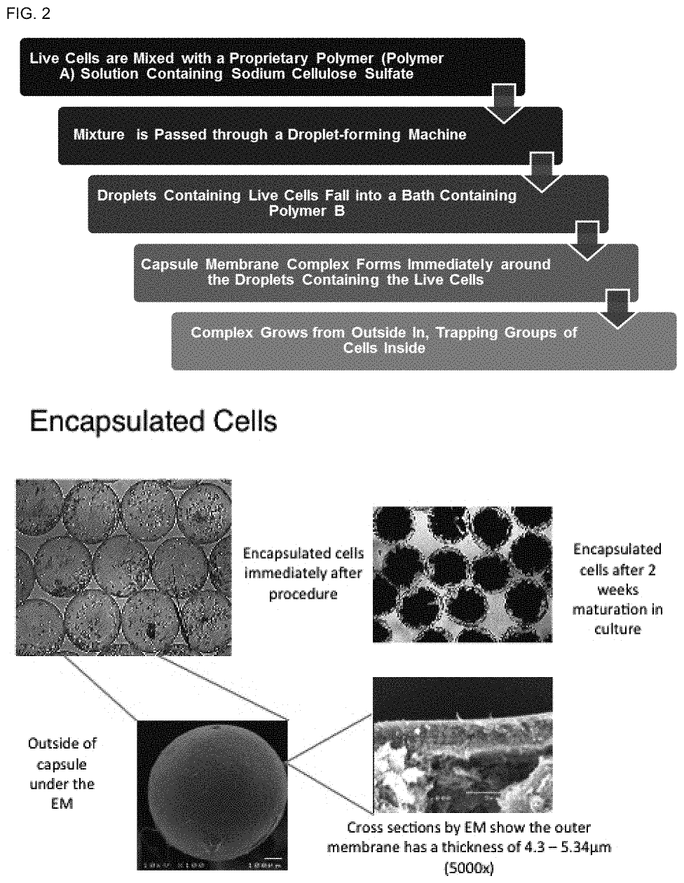

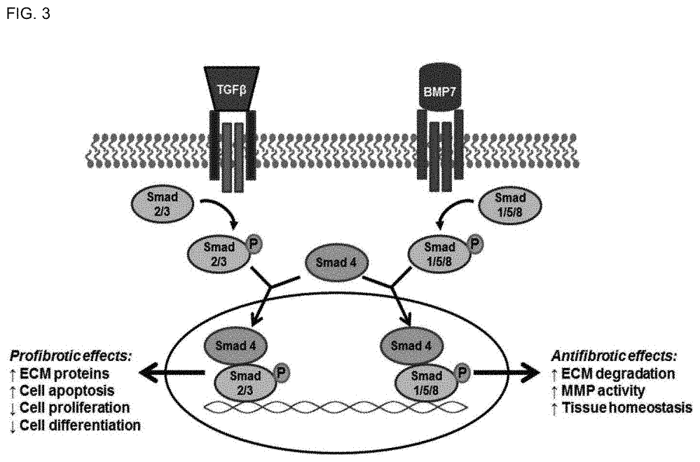

An exemplary encapsulation process is shown in FIG. 2. In an embodiment, the live cells are first suspended in a medium that contains a polymer and sodium cellulose sulfate. This suspension is passed through a droplet-forming machine, and the resulting droplets fall into a solution containing another polymer. As the two polymers interact, immediately a membrane forms around each droplet. This develops into a shell around the droplet, resulting in a spherical capsule 0.7-0.8 mm in diameter. In embodiments, the size of the capsule can be, for example, between 50 μm and 1000 μm, between 100 μm and 800 μm, between 100 μm and 600 μm, between 150 μm and 600 μm, between 200 μm and 600 μm, between 300 μm and 600 μm, between 300 μm and 500 μm, between 300 μm and 400 μm, or the like.

Disclosed embodiments provide precise treatments by enabling injected encapsulated BMP7-producing cells to be administered in the area where treatment is desired. This precision can be of increased importance with BMP7, as BMP7 has been implicated in various types of cancer, particularly prostate cancer and breast cancer.

For treatment of disease, for example pancreatic cancer, CKD, or fibrosis, each capsule contains about 10,000 living cells. This number of live cells can differ depending upon the size of the cells encapsulated and for what purpose they are to be used. For example, the number of living cells per capsule can be 1000 cells, 2000 cells, 3000 cells, 4000 cells, 5000 cells, 6000 cells, 7000 cells, 8000 cells, 9000 cells, 10,000 cells, 11,000 cells, 12,000 cells, 13,000 cells, 14,000 cells, 15,000 cells, 16,000 cells, 17,000 cells, 18,000 cells, 19,000 cells, 20,000 cells, or more. In embodiments, the number of living cells per capsule can be at least 1000 cells, at least 2000 cells, at least 3000 cells, at least 4000 cells, at least 5000 cells, at least 6000 cells, at least 7000 cells, at least 8000 cells, at least 9000 cells, at least 10,000 cells, at least 11,000 cells, at least 12,000 cells, at least 13,000 cells, at least 14,000 cells, at least 15,000 cells, at least 16,000 cells, at least 17,000 cells, at least 18,000 cells, at least 19,000 cells, at least 20,000 cells, or more. In embodiments, the number of living cells per capsule can be not more than 1000 cells, not more than 2000 cells, not more than 3000 cells, not more than 4000 cells, not more than 5000 cells, not more than 6000 cells, not more than 7000 cells, not more than 8000 cells, not more than 9000 cells, not more than 10,000 cells, not more than 11,000 cells, not more than 12,000 cells, not more than 13,000 cells, not more than 14,000 cells, not more than 15,000 cells, not more than 16,000 cells, not more than 17,000 cells, not more than 18,000 cells, not more than 19,000 cells, not more than 20,000 cells, or more.

In disclosed embodiments, the live encapsulated cells can be frozen and later thawed with viability of approximately 50%, 60%, 70%, 80%, 90%, or more, upon thawing. Thus, the frozen encapsulated cells have a long-term (5+ years) shelf life which results in more manageable logistics. In embodiments, capsules are made principally of cellulose, a bio-inert material in the human body. Further embodiments can comprise capsules of collagen, chitosan, gelatin, agarose, combinations thereof, and the like.

In embodiments, the capsules are durable, resilient and long-lasting. The properties of disclosed capsules allows them to be implanted in the human body by using needles or catheters without damage to the human body. In embodiments, disclosed capsules do not degrade, even after being present in the body for over two years. The capsules do not cause any damage to or inflammation of tissues in the body.

In embodiments, the cell type chosen depends on the desired application of the cell microcapsules. The encapsulated cells can be from the patient (autologous cells), from another donor (allogeneic cells) or from other species (xenogeneic cells). Depending on the application, the cells can be genetically altered to express any desired protein.

Exemplary embodiments include a method for treating a tissue or organ in a subject in need thereof comprising the step of administering a therapeutically effective amount of encapsulated cells to the tissue or organ.

The therapeutic methods disclosed herein can be used to treat conditions including, but not limited to, thickening and scarring of connective tissue, or other damage to tissues and organs which were the result of injury, trauma, non-trauma, surgery, hereditary disease, or other chronic or non-chronic conditions. In still other embodiments, disclosed methods can be employed to treat conditions such as adhesive capsulitis, arterial fibrosis, arthrofibrosis, Crohn's disease, cirrhosis, cystic fibrosis, Dupuytren's contracture, demyelination and related demyelination conditions; endomyocardial fibrosis, fibroleiomyoma, fibromyoma, idiopathic pulmonary fibrosis, keloid, mediastinal fibrosis, multiple sclerosis, myelofibrosis, myoma, nephrogenic systemic fibrosis, old myocardial infarction, Peyronie's disease, progressive massive fibrosis, pulmonary fibrosis, retroperitoneal fibrosis, scleroderma/systemic sclerosis, uterine fibroids, uterine leiomyoma, other conditions relating to excessive connective tissue, and combinations thereof.

Further uses of BMP7 contemplated herein include:

| Fibrotic disease | Treatment | Findings |
|---|---|---|
| [symbol] | [symbol] | [symbol] |
| | [symbol] | [symbol] |
| [symbol] | [symbol] | [symbol] |
| | [symbol] | [symbol] |
| | [symbol] | [symbol] |
| | [symbol] | [symbol] |
| [symbol] | [symbol] | [symbol] |
| | [symbol] | [symbol] |
| | [symbol] | [symbol] |
| [symbol] | [symbol] | [symbol] |

[symbol]

indicates data missing or illegible when filed

Treatment models using BMP7 include:

| Animal model | Species | Disease model | Effect of BMP-7 |
|---|---|---|---|
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |

[symbol]

indicates data missing or illegible when filed

In embodiments, the area or tissue to be treated can be an organ, joint, or other area of the body with fibroids or excessive connective or scar tissue. In other embodiments, the treated organ can be, but is not limited to, the kidney, liver, heart, lung, skin, intestine, or uterus.

The disclosed methods can also comprise the co-administration of bioactive agents with the encapsulated cells. By “co-administration” is meant administration before, concurrently with, e.g., in combination with bioactive agents in the same formulation or in separate formulations, or after administration of a therapeutic composition as described above.

As used herein, the phrase, “bioactive agents” refers to any organic, inorganic, or living agent that is biologically active or relevant. For example, a bioactive agent can be a protein, a polypeptide, a nucleic acid, a polysaccharide (e.g., heparin), an oligosaccharide, a mono- or disaccharide, an organic compound, an organometallic compound, or an inorganic compound. It can include a living or senescent cell, bacterium, virus, or part thereof. It may include a biologically active molecule such as a hormone, a growth factor, a growth factor producing virus, a growth factor inhibitor, a growth factor receptor, an anti-inflammatory agent, an antimetabolite, an integrin blocker, or a complete or partial functional sense or antisense gene, including siRNA. It can also include a man-made particle or material, which carries a biologically relevant or active material. An example is a nanoparticle comprising a core with a drug and a coating on the core.

Bioactive agents can also include drugs such as chemical or biological compounds that can have a therapeutic effect on a biological organism. Non-limiting examples include, but are not limited to, growth factors, anti-rejection agents, anti-inflammatory agents, anti-infective agents (e.g., antibiotics and antiviral agents), and analgesics and analgesic combinations. Anti-inflammatory agents can be useful as additional agents to counteract the inflammatory aspects of the fibrotic process.

Combinations, blends, or other preparations of any of the foregoing examples can be made and still be considered bioactive agents within the intended meaning herein. Aspects of the present disclosure directed toward bioactive agents can include any or all of the foregoing examples. In other embodiments, the bioactive agent can be a growth factor. A growth factor is any agent which promotes the proliferation, differentiation, and functionality of the implanted stem cell. Non-limiting examples of suitable growth factors may include, but are not limited to, leukemia inhibitory factor (LIF), epidermal growth factor (EGF), fibroblast growth factor (FGF), insulin-like growth factor (IGF), vascular endothelial growth factor (VEGF), human growth hormone (hGH), platelet-derived growth factor (PDGF), interleukins, cytokines, and/or combinations thereof.

In some embodiments, the bioactive agent can be an immunosuppressive agent. An immunosuppressive agent is any agent which prevents, delays the occurrence of, or decreases the intensity of the undesired immune response, e.g., rejection of a transplanted cell, tissue, or organ, or graft-versus-host disease. Preferred are immunosuppressive agents which suppress cell-mediated immune responses against cells identified by the immune system as non-self. Examples of immunosuppressive agents can include, but are not limited to, cyclosporin, cyclophosphamide, prednisone, dexamethasone, methotrexate, azathioprine, mycophenolate, thalidomide, FK-506, systemic steroids, as well as a broad range of antibodies, receptor agonists, receptor antagonists, and other such agents as known to one skilled in the art. In other embodiments, bioactive agents that may be administered include anti-fibrotic agents including, but not limited to, nintedanib, INT-767, emricasan, VBY-376, PF-04634817, EXC 001, GM-CT-01, GCS-100, Refanalin, SAR156597, tralokinumab, pomalidomide, STX-100, CC-930, simtuzumab, anti-miR-21, PRM-151, BOT191, palomid 529, IMD1041, serelaxin, PEG-relaxin, ANG-4011, FT011, pirfenidone, F351 (perfenidone derivative), THR-184, CCX-140, FG-3019, avosentan, GKT137831, PF-00489791, pentoxifylline, fresolimumab, and LY2382770.

### EXAMPLES

### Example 1

### Overexpression of BMP7 (Ad-BMP7) Effect on TGFβ

Adenovirus-mediated expression of BMP7 suppresses the development of liver fibrosis in rats. Liver cirrhosis, which is caused by the accumulation of extracellular matrix materials, is a serious clinical problem that can progress to hepatic failure. Transforming growth factor-β (TGFβ) plays a pivotal role in extracellular matrix production, but bone morphogenetic protein (BMP)-7, a member of the TGFβ superfamily, can antagonize the fibrogenic activity of TGFβ.

In this study, we examined whether adenovirus-mediated overexpression of BMP7 (Ad-BMP7) antagonized the effect of TGFβ in vitro and in vivo.

In primary cultured rat stellate cells and the LX-2 human stellate cell line, induction of BMP7 by Ad-BMP7 infection decreased the expression of collagen 1A2 mRNA and smooth muscle α-actin in the presence or absence of TGFβ, via Smad 1/5/8 phosphorylation. BMP7 triggered the mRNA expression of inhibitors of differentiation 2 (Id2) in LX-2. Although endogenous expression of BMP7 was hardly detectable, Smad1 and Id2 overexpression increased BMP7 expression in LX-2. A liver fibrosis model was induced by the repetitive intraperitoneal injection of thioacetamide (200 mg/kg body weight) twice per week for up to 7 weeks. In rats administered Ad-BMP7 via the tail vein, hydroxyproline content and the areas stained by Sirius red dye in the liver were significantly reduced compared to controls. Ad-Id2 also reduced fibrosis. These data demonstrate that BMP7, Smad 1/5/8 and Ids interact to antagonize hepatic fibrogenesis

Encapsulated BMP7 positive cells were tested to measure in-vitro expression of BMP7 protein with ELISA after encapsulation to make sure BMP7 can be excreted and measured out side of cellulose capsule.

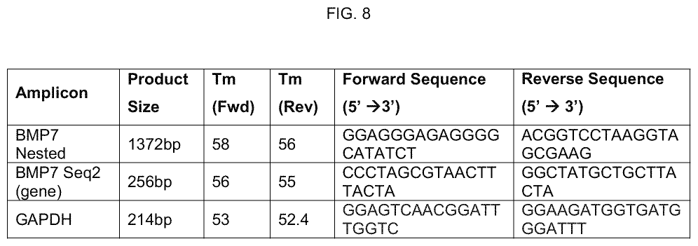

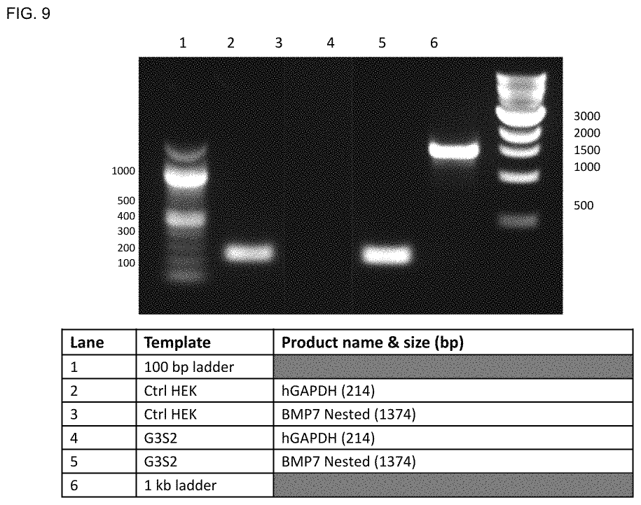

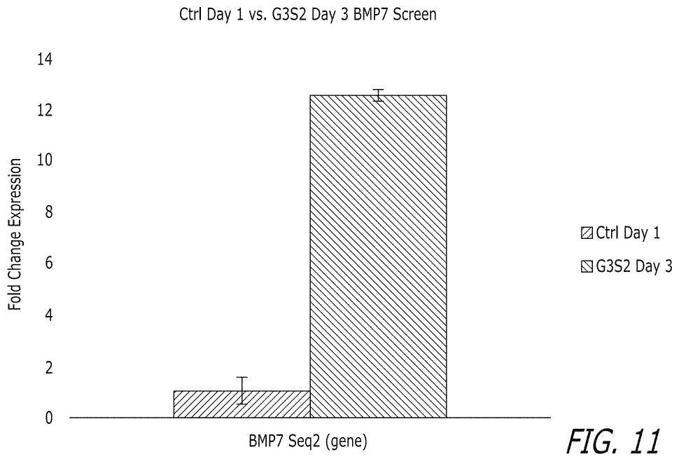

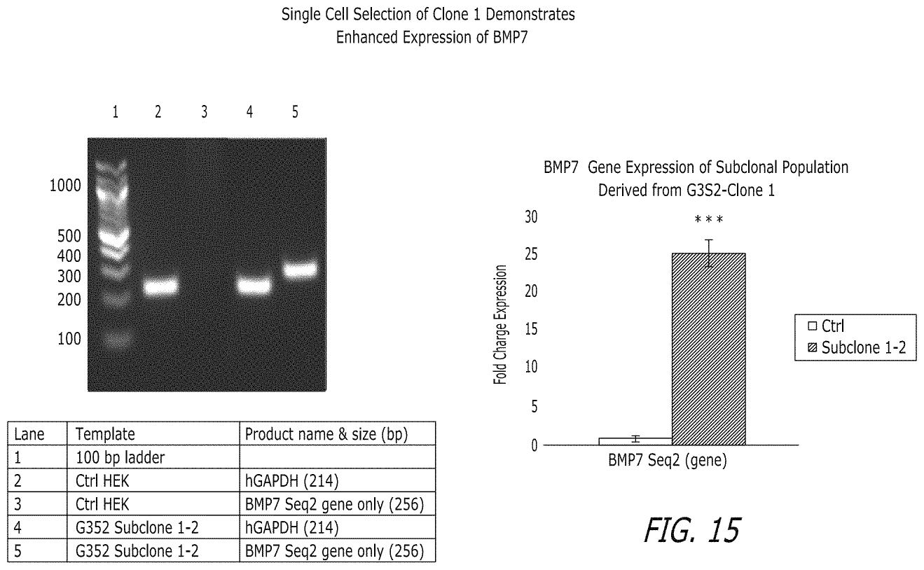

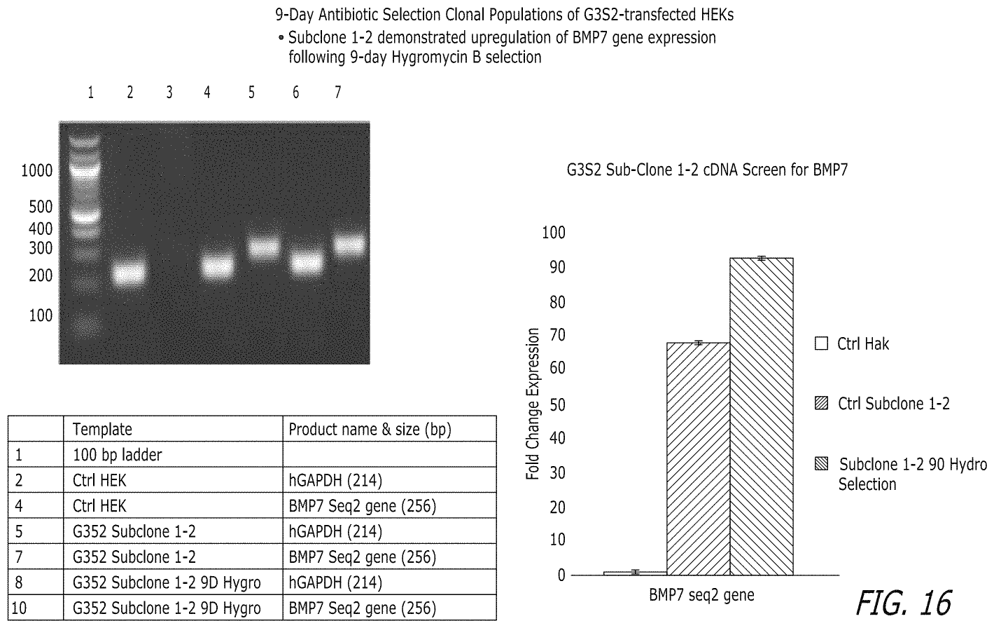

These tests are reflected in FIGS. 8-19.

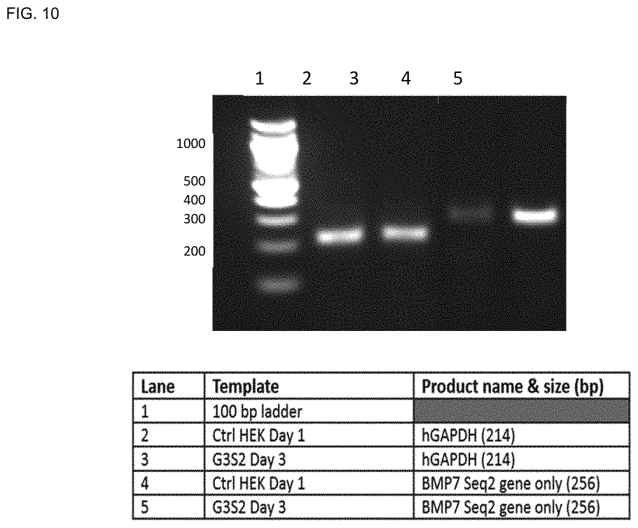

First, Feline BMP7 gene sequence was integrated into Genie-3, Sequence-2 (G3S2) plasmid design. Next, hygromycin selection was performed, then an HEK293T cell line was transfected with the plasmid (FIG. 10). Feline BMP7 has high homology to Human BMP7, over 94%, and can be detected using both Feline and Human BMP7 ELISA kits. The GENIE BMP7 construct also included a suicide gene (FKBP Caspase 9). The chemical activator used to activate the FKBP Caspase 9 gene is a chemical inducer called AP1903 or AP20187.

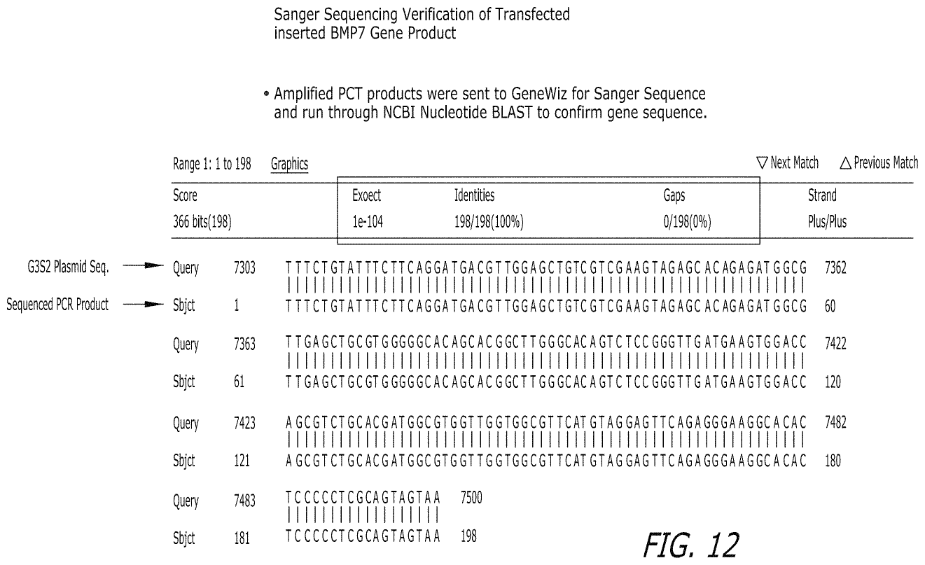

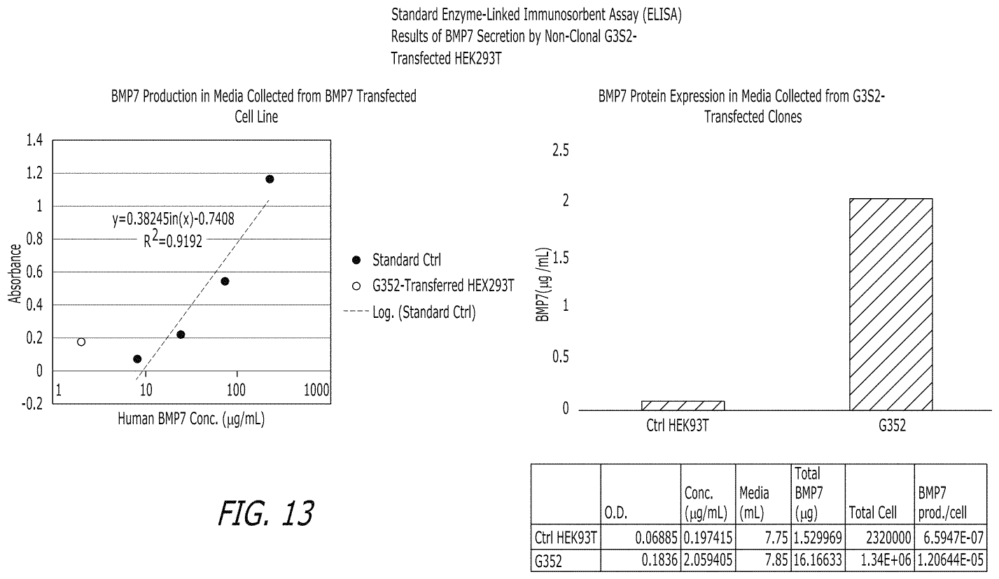

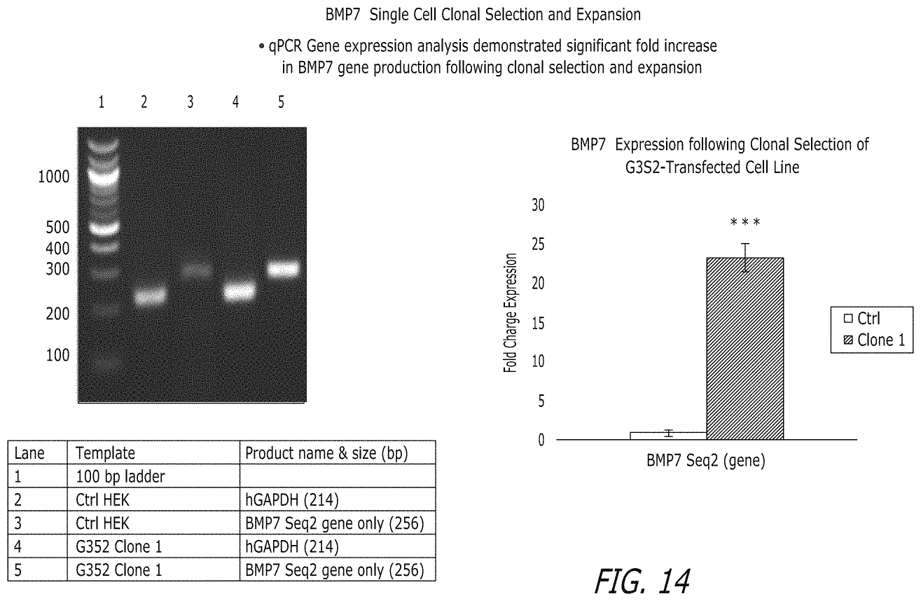

Then BMP7 transfection was performed, and hygromycin selection was performed on the cell line. BMP7 expression of these cells is shown in FIG. 12. Gene expression is shown in FIG. 13. FIG. 14 shows sequencing verification of the transfected inserted gene.

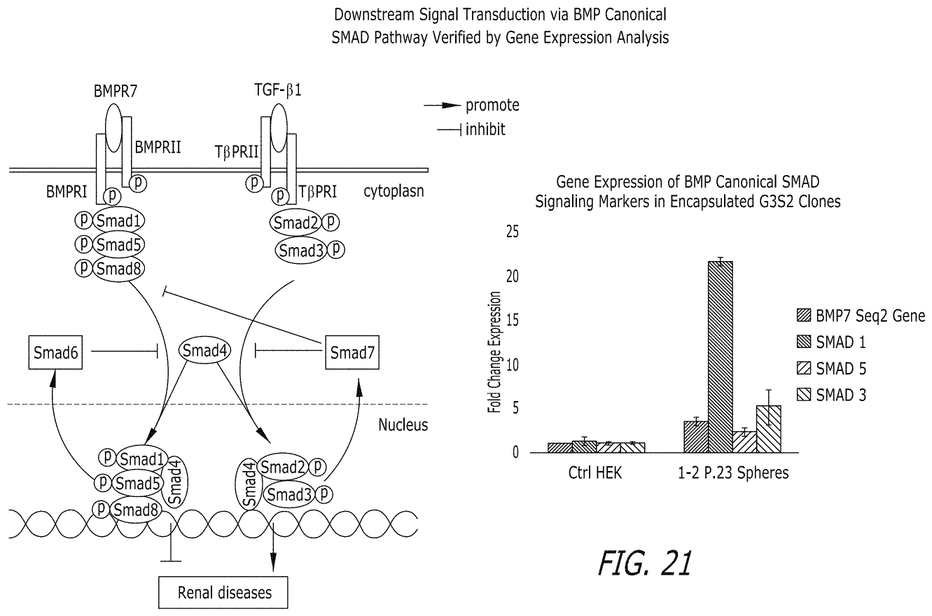

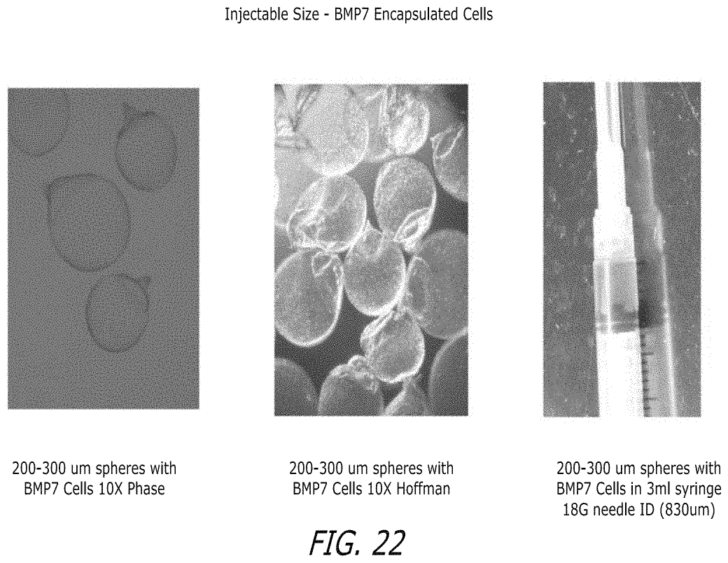

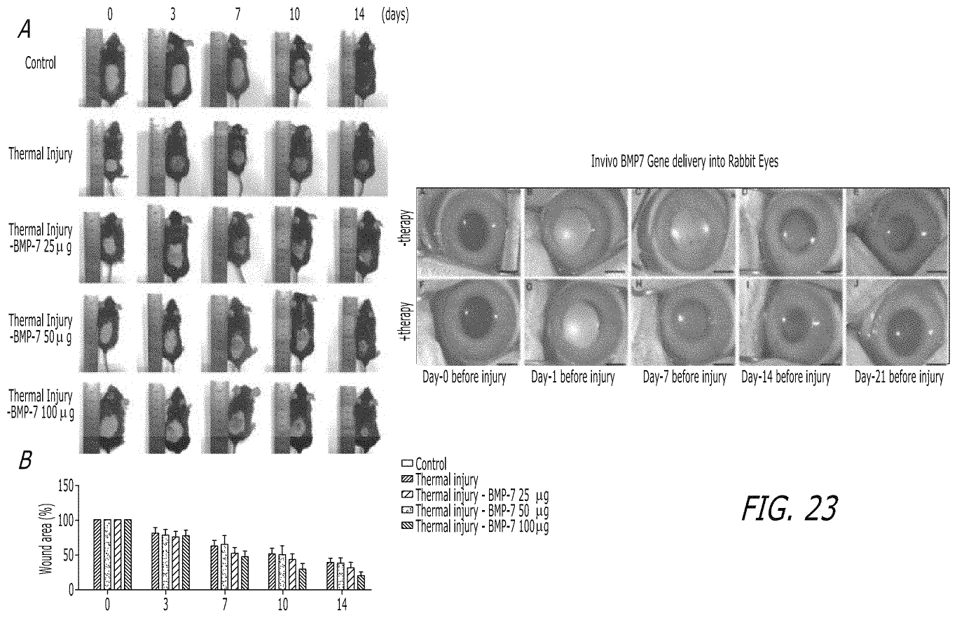

Next, single cell clonal selection was performed (FIG. 21), followed by expansion.

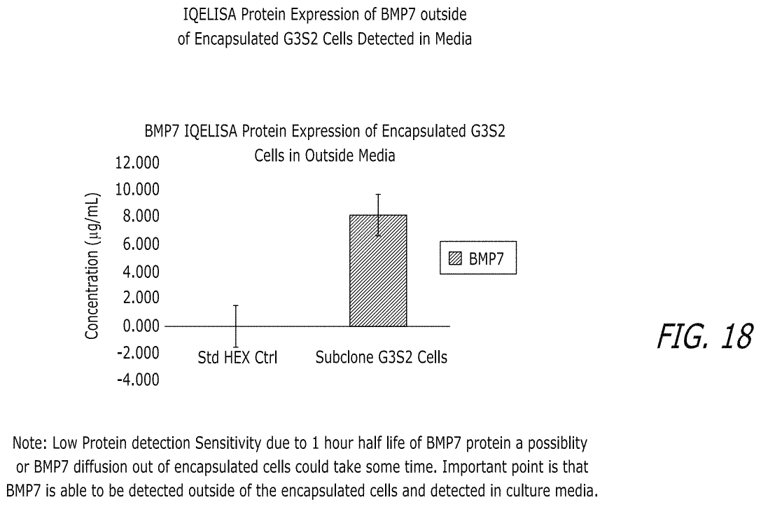

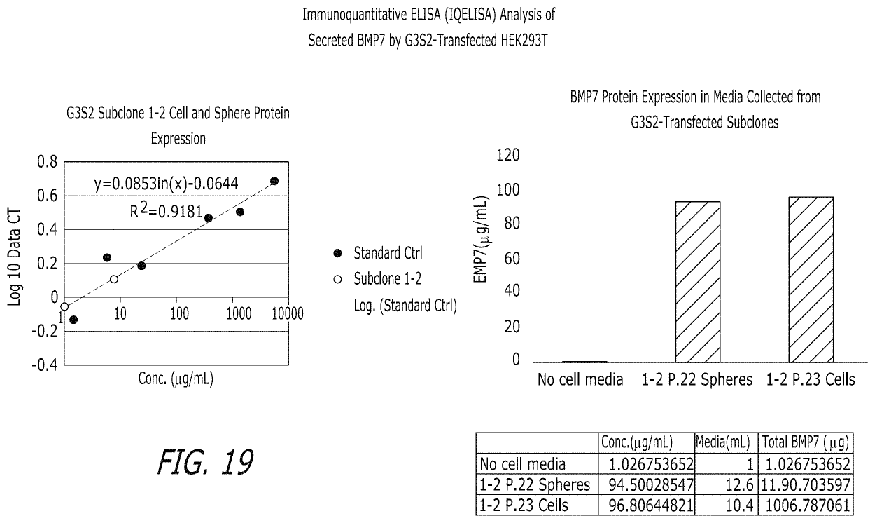

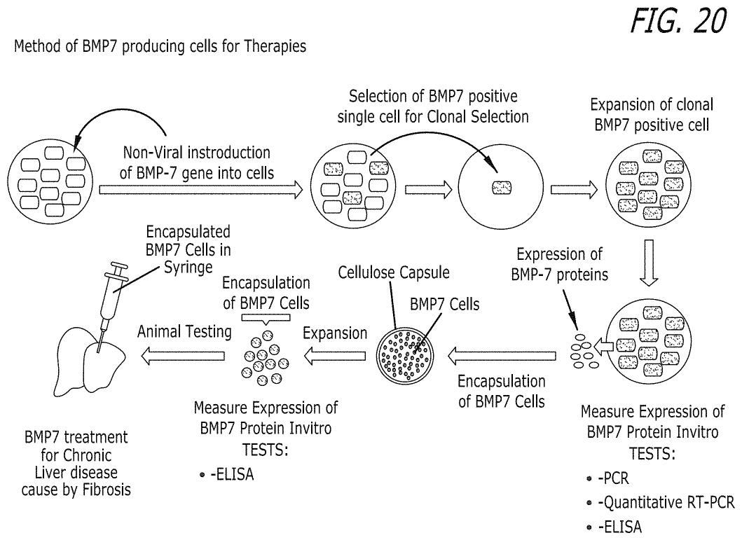

The cells were then encapsulated (FIG. 18). FIG. 19 shows IQELISA results for BMP7 expression outside of the encapsulated cells.

### Example 2

### Effect of Freeze/Thaw Cycles on BMP7 Expression

Expression of BMP7 from Encapsulated HEK cells was measured before encapsulation, after encapsulation, after freezing/thawing of encapsulated cells. All conditions were able to express high amounts of BMP7 into the media outside the encapsulated cells vs control HEK cells and vs culture media alone.

### Example 3

### Long-Term Effects on Mice

Encapsulated BMP7 producing cells (circumference size ˜300-500 um) were loaded into a 3 ml syringe with 18 G needle ˜10-20 encapsulated cells were injected into each hind thigh of C57B6 mice. ˜100 encapsulated cells were also injected intraperitoneally into C57B6 mice. After 7 months mice were sacrificed. No visual signs of tumors or abnormalities in areas of injections or organ before or after sacrificing injected mice for necropsy. Mice showed no adverse reaction to injections during the 7 months after injection and were visually healthy before sacrificing.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method for treating a tissue or organ in a subject in need thereof comprising administering a therapeutically effective amount of encapsulated transfected cells to the tissue or organ, thereby improving the function of the tissue or organ, wherein said transfected cells express BMP7.

2. A method for treating fibrosis in a subject in need thereof comprising the steps of:
(a) detecting the level of fibrosis in the tissue or organ of the subject;
(b) administering a therapeutically effective amount of transfected encapsulated cells to the tissue, wherein said cells express BMP7;
thereby reducing fibrosis and improving the function of the tissue or organ.

3. The method of claim 2, wherein the level of fibrosis is detected using an imaging method selected from computed tomography (CT), magnetic resonance imaging (MRI), ultrasound, and optical tomography.

4. The method of claim 2, wherein step (a) further comprises the steps of:
(a) obtaining a biological sample from the subject;
(b) determining the amount of at least one marker of fibrosis in the biological sample;
(c) comparing the amount of the at least one marker of fibrosis to a reference value.

5. The method of claim 4, wherein the reference value is the amount of the at least one marker of fibrosis in a biological sample obtained from the subject prior to step (a), and wherein a reduction in the amount of the at least one marker of fibrosis as compared to the reference value indicates a reduction of fibrosis in the tissue or organ of the subject.

6. The method of claim 4, wherein the reference value is the amount of the at least one marker of fibrosis in a biological sample from a subject or subjects known to have fibrosis, and wherein a reduction in the amount of the at least one marker of fibrosis as compared to the reference value indicates a reduction of fibrosis in the tissue or organ of the subject.

7. The method of claim 6, wherein the biological sample is blood, plasma, serum, urine, or tissue.

8. The method of claim 4, wherein the biological sample is a sample from a tissue or organ.

9. The method of claim 6, wherein the at least one marker of fibrosis is urea, creatinine, or blood urea nitrogen.

10. The method of claim 6, wherein the at least one marker of fibrosis is fibroblast-specific protein 1 (FSP-1), [symbol] -smooth muscle actin ( [symbol] -SMA), interleukin 6 (IL-6), monocyte chemotactic protein-1 (MCP-1), transforming growth factor [symbol] 1 (TGF- [symbol] 1), or Smad3.

11. The method of claim 10, wherein the subject is a mammal.

12. The method of claim 10, wherein the subject is a non-human primate.

13. The method of claim 11, wherein the subject is a human.

14. The method of claim 1, wherein the method treats a condition selected from the group consisting of adhesive capsulitis, arterial fibrosis, arthrofibrosis, Crohn's disease, cirrhosis, cystic fibrosis, Dupuytren's contracture, endomyocardial fibrosis, fibroleiomyoma, fibromyoma, idiopathic pulmonary fibrosis, keloid, mediastinal fibrosis, myelofibrosis, nephrogenic systemic fibrosis, old myocardial infarction, myoma, Peyronie's disease, progressive massive fibrosis, pulmonary fibrosis, retroperitoneal fibrosis, scleroderma/systemic sclerosis, uterine fibroids, and uterine leiomyoma.

15. A method of treating fibrosis of the liver, kidney, spleen, lungs, or heart, comprising administering to a fibrotic organ encapsulated cells that produce human BMP7.
